(19) United States
(12) Patent Application Publication
(10) Pub. No.: US 2008/0228240 A1
(43) Pub. Date: Sep. 18, 2008

(54) Long Term Bi-Directional Axon-Electronic Communication System

(76) Inventors: David J. Edell, Lexington, MA (US); Ronald R. Riso, Somerville, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/629,257

(22) PCT Filed: Jun. 15, 2005
(86) PCT No.: PCT/US05/21081

§ 371 Date: Dec. 12, 2006

Related U.S. Application Data
(60) Provisional application No. 60/580,426, filed on Jun. 17, 2004.
(60) Provisional application No. 60/668,401, filed on Apr. 5, 2005.
(60) Provisional application No. 60/675,570, filed on Apr. 28, 2005.

(51) Int. Cl. A61N 1/05

(52) U.S. Cl. 607/48; 607/60

(57) ABSTRACT

A long term bi-directional axon-electronic communication system that provides signaling capability at the level of individual nerve fascicles, bundles of axon and even axons is disclosed. The bi-directional communication system is a modular approach for achieving a chronic enduring interface to peripheral or central nerve atoms for the purpose of restoring function to disabled persons or animals with sensory and/or motor impairments. One embodiment of the communication system includes a multi-channeled nerve-muscle graft chamber for making the nerve-muscle connection. Another embodiment includes a regeneration based microtube nerve interface for bi-directional communication. The interface communication system permits amputees to obtain simultaneous control of multi-degree of freedom powered prostheses by means of naturally produced neural activity from the stamps of the amputated nerves in their residual limbs.

### CROSS REFERENCE TO RELATED APPLICATIONS

This application claims priority from U.S. Provisional Application Nos. 60/580,426, filed Jun. 17, 2004; 60/668,401, filed Apr. 5, 2005; and 60/675,570, filed Apr. 28, 2005, the whole of which are hereby incorporated by reference herein.

### STATEMENT REGARDING FEDERALLY SPONSORED RESEARCH OR DEVELOPMENT

N/A

### BACKGROUND OF THE INVENTION

In the field of rehabilitation of humans or other mammals following an accident or other injury, one of the more intractable problems has been how to restore the connections of injured nerves to their previously associated, or replacement, tissue, e.g. for active movement or for sensory perception or feedback, at anything like their previous sensitivity. For example, control of multiple degree of freedom myo-electric prosthetic arms by amputees who have above elbow transections is limited because of a lack of independently recordable electromyographic (EMG) signals from the residual limb muscles. Typically, reciprocal commands are derived using one pair of pickup electrodes to register activity from the biceps while a second pair of electrodes is used to detect triceps activity. These two muscles can control hand opening and closing or, by the operation of various mode switching mechanisms, they can be toggled to operate wrist or elbow rotation. The task of performing mode switching is tedious since it breaks up any compound arm/hand movement into serial positioning steps.

A proposed solution to this problem that has been long sought after is the capability of connecting to individual motor nerve fascicles of the major upper arm trunk nerves. Examples of competing approaches for nerve interface technologies include: intrafascicular, regeneration, sieve, penetrating brush arrays and cuff devices. Examples of these devices are shown in FIG. 1. Thus far, however, no designs have been demonstrated to be entirely satisfactory. Aside from issues of positional stability, safety and signal selectivity, the very low intensity signals that can be recorded directly from nerves are easily disrupted by electrical noise present in the environment from the electric motors used in appliances (e.g., elevators, door openers) and from telecommunications equipment. Given the present explosion in consumer demand for wireless devices, electrical interference issues are likely only to intensify. Further, amputated nerves tend to degenerate to varying degrees unless in contact with healthy tissues such as muscle and skin, or under some circumstances which are not well understood, connective tissue and fat.

An alternative strategy attempted has been to take transected motor nerves and graft them to existing healthy muscle tissue so that the nerves can grow into and form functional connections with the host muscle. Voluntary activation of the nerve can then be detected by registering the EMG that is produced within the host muscle. The muscle is thus used as a neural signal amplifier to convert the nerve activity into EMG (electromyographic) activity.

This strategy has been studied with a few human upper extremity amputees. The subjects were observed to voluntarily activate the grafted nerve-muscle units to produce EMG signals that could be sensed and used for prosthetic control. By activating the four grafted nerves individually, the amputee individual was able to produce different levels of contraction in each of the four muscle compartments. Moreover, the independent signals were able to control both hand and elbow prosthesis functions independently.

However, this early technology for creating nerve interfaces for EMG control has suffered from several problems. In particular, one drawback of transferring an amputated nerve stump to a normal muscle is that the normal innervation of the host muscle must be permanently removed so that only the grafted nerve is capable of evoking EMG activity. In doing this the original function of the host muscle is sacrificed in order to create the interface to the grafted nerve. A second drawback is that the host muscle becomes innervated by the mix of nerve fascicles that were originally targeted at multiple other muscles and thus muscle specificity is immediately compromised. A third drawback is that the muscle sites that can be candidates to receive grafted nerves are limited to those muscles which are physically positioned so that the nerve that is to be grafted can be brought to the muscle. Furthermore, this approach relies on surface electrode recordings which are inherently unstable and unspecific with regard to the registration of EMG activity due to “cross over” activity from nearby or underlying muscles.

### BRIEF SUMMARY OF THE INVENTION

The present invention, directed to a long term bi-directional axon-electronic communication system that provides signaling capability at the level of individual nerve fascicles, bundles of axon and even axons advances the technology of nerve interfaces for EMG control by removing the drawbacks of the prior art. The bi-directional communication system according to the invention represents a modular approach to achieving a chronic enduring interface to peripheral or central nerve axons for the purpose of restoring function to disabled persons or animals with sensory and/or motor impairments. Two particularly preferred embodiments of a small implantable bi-directional axon-electronic interface system with associated telemetry according to the invention are described in detail herein. Depending on the specific application, these two embodiments can be implemented either individually or together. One embodiment includes a multi-channeled nerve-muscle graft chamber for making the nerve-muscle connection. The second embodiment includes a regeneration based microtube nerve interface for bi-directional communication. The utility of the invention is illustrated with particular application to the problem of the control of prosthetic limbs, and the system will allow amputees to obtain simultaneous control of multi-degree of freedom powered prostheses by means of naturally produced neural activity from the stumps of the amputated nerves in their residual limbs. The approach is based on the physiological fact that the motor nerves in an amputee's residual limb remain capable of being activated by the amputee and can even evoke muscular contractions when they succeed in re-establishing connections to muscle tissue. This can be achieved, for example, by using muscle-nerve grafting techniques whereby the stump of an amputated nerve is grafted onto a host muscle, muscle tissue or fragment thereof. Additionally, the sensory afferent nerve fibers in the amputee's residual limb nerves retain functional connectivity to the amputee's brain and, if activated by electrical stimulation or by mechanical means, are capable of evoking sensory experiences. With appropriately controlled activation of the sensory fibers, meaningful sensory feedback information regarding the state of the prosthetic limb can be provided to the prosthesis user.

The advantages of the system of the invention include the capability of recording from or stimulating every nerve fiber (axon) in a targeted nerve fascicle or group of nerve fascicles. This level of fiber selectivity in an interface that is stable over extended periods, such as the months and years that are needed for clinical applications, has not been achievable thus far using any prior art technology.

### BRIEF DESCRIPTION OF THE DRAWINGS

Other features and advantages of the invention will be apparent from the following description of the preferred embodiments thereof and from the claims, taken in conjunction with the accompanying drawings, in which:

FIG. 1 shows various prior art devices for achieving a chronic interface to peripheral nerves; A) polyimide based sieve electrode (T. Steiglitz et al. [1997]); B) silicon slotted sieve array (Edell et al. [1986]); C) brush style slant array (R. Norman—Bionic Technology Inc.); D) Platinum microwire array (Edell et al. [1996]);

FIG. 2 shows one preferred embodiment of the long term bi-directional axon-electronic communication system according to the invention;

FIG. 3 shows an alternative design for electrode contacts in the communication system of FIG. 2;

FIG. 4 shows another preferred embodiment of the long term bi-directional axon-electronic communication system according to the invention;

FIG. 5 shows a sensory feedback actuator system for use with the communication system of FIG. 2; and

FIG. 6 is a graph showing signal amplitude obtainable versus microtube diameter for microtubes one millimeter in length in the communication system of FIG. 4.

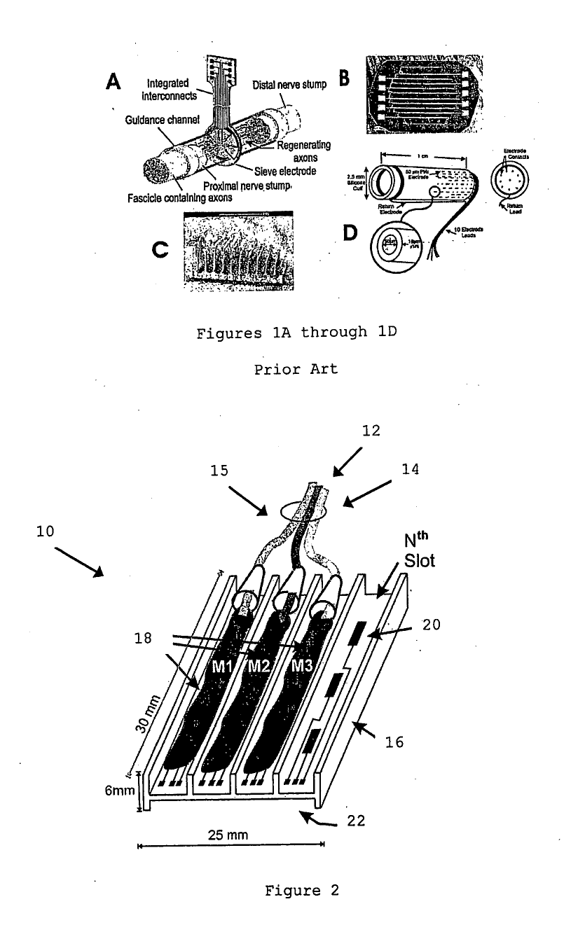

### DETAILED DESCRIPTION OF THE INVENTION

The long term bi-directional axon-electronic communication system according to the invention ensures that an adequate compliment of independently accessible efferent and afferent nerve fibers will be obtained for effective functionality. It is then possible, for example, to interface individually with any arbitrary motor or sensory nerve that can be surgically exposed and, moreover, with the axons of each fascicle present within that nerve. This can greatly increase the available number, e.g., of control signal sources that the amputee can produce without compromising existing healthy muscle. Perhaps of greater significance, the neural control can be more natural in that the same function previously served by a particular motor nerve fascicle can be re-directed to the analogous prosthetic actuator. The major advantage of using nerve-muscle grafts as a command source for prosthetics control is that the muscle acts like a biological amplifier that transforms the weak nerve activity (typically only a few microvolts in amplitude) into EMG activity, which is hundreds of times stronger and thus more reliably sensed using recording electrodes. A second advantage is that the EMG signal from each muscle can be used to control a different prosthesis function, such as wrist rotation versus finger closing, so that more simultaneous prosthetic hand control could be obtained, instead of present control systems which are based on sequential control schemes.

The system of the invention will lead, for example, to a more universal nerve-muscle replant technique: the muscle tissue that will be placed into the implanted nerve interface chamber may consist, e.g., of autologos muscle slices or of muscle tissue that has been grown using muscle precursor cells. In a preferred embodiment, the implanted chamber will be fitted with “on board” amplifier and telemetry circuitry, which will provide exceptionally clear and stable EMG recordings in comparison to the use of surface EMG recording. The telemetry system can be, e.g., based on RF, optical or ultrasound transmissions, and powered, e.g., by batteries, RF transmission, optical power or other means.

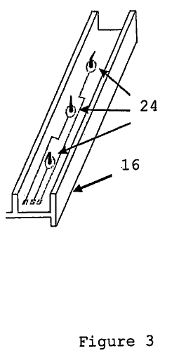

The approach utilized by the present invention is illustrated in FIG. 2. Referring to FIG. 2, a multi-channeled nerve-muscle graft chamber 10 for making the nerve-muscle connection includes a nerve cuff 14 for enclosing an input nerve 12, e.g., a nerve transected by amputation, and holding in position, separated peripheral nerve fascicles 15. The fascicles from the input nerve are allowed to grow into a receptacle chamber 16 that contains, e.g., small slices 16 of autologus muscle or pieces of muscle taken from the amputated muscle remains, or from healthy muscles. Alternatively, the muscle tissue may be derived from muscle precursor or other cells. Once the nerve regenerates and innervates the muscle tissue, the pieces of muscle tissue will effectively become biological amplifiers for the nerve signals. Small blood vessel from the nerve or nearby tissue will grow into the target tissue. Also, the nerve and muscle will exchange trophic factors to maintain the viability of both the muscle and the nerve.

Referring still to FIG. 2, the basic structure of the receptacle chamber 16 can be molded from silicone or other biocompatible polymer. All edges are smooth and rounded. The electrode contacts 20 may consist, e.g., of small pieces of platinum or other biocompatible conductors, or they may consist of conductive films applied to polymer substrates such as Liquid Crystal Polymer or polyimide. Referring to FIG. 3, the electrode contacts 24 can also consist of short needles attached to the bottom or sides of the individual chamber compartments in such a way that they impale the muscle tissue (as with a “bed of nails”), and such an arrangement can also be helpful in anchoring the muscle tissue.

Referring again to FIG. 2, the size of the chamber is small (e.g., 25×30×6 mm) so that it can be implanted near the locations of the most appropriate nerves (such as the median, radial, musculocutaneous and ulnar nerves of the arm). When a lead out cable is used, it can be connected to an external telemetry module. Where the muscle-nerve chamber can be located just beneath the skin, the telemetry system 22 would be fabricated to be integral with the chamber—entirely eliminating the need for even a short lead out cable. Besides not having to sacrifice the function of the host muscle, the use of the special chamber with telemetry can lead to more consistency in the recording of the EMG signals and less channel cross-talk by eliminating the need to use surface EMG recording techniques.

While it is known that there is variable mixing of efferent fibers from different muscles in fascicles, depending primarily on the distance from the target muscle, in general, the majority of efferent fibers in a given fascicle are from a single target muscle, which would dominate the response. Since each fascicle will be isolated to a single muscle compartment, its recorded activity would then be expected to be predominantly from information previously targeted at a specific amputated muscle. By simple threshold discrimination, it will be possible to obtain high quality control signals targeted at a single muscle. EMG recording channels (represented by the different compartments within the multi-compartmented chamber) that do not produce distinct, independent signals would simply be ignored initially, as many more channels of information would be available than can be taken advantage of by existing prostheses. In later developments, as more functional prosthetic limbs become available that will take advantage of this rich supply of high quality, stable, control signals, signal processing schemes employing artificial neural networks, or fuzzy logic techniques, for example, could be applied to extract additional channels of information as needed.

Likewise, predominantly afferent fascicles can be interfaced with segments of skin to maintain the afferent fibers by trophic factors released from cutaneous sensors. By incorporating electrodes in the nerve-skin graft chamber, portions of the nerve can be stimulated to provide crude but useful cutaneous sensations.

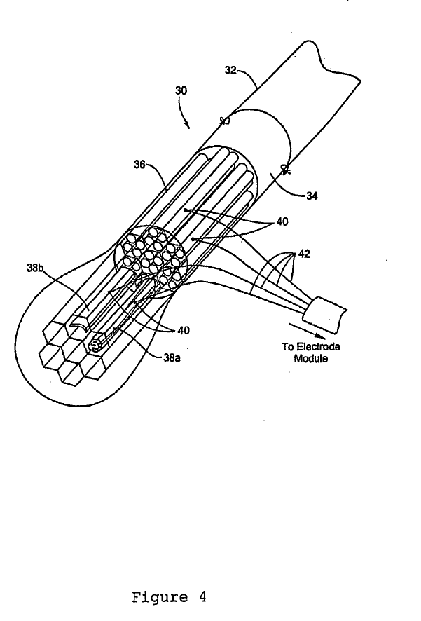

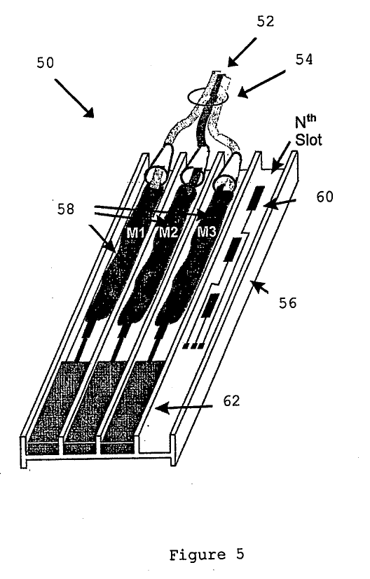

To further ensure that an adequate compliment of independently accessible efferent and afferent nerve fibers will be obtained with the nerve interface, in another aspect, a particularly preferred embodiment of the present invention is to provide a microtube based regeneration electrode interposed between each isolated transected nerve fascicle and the nerve-muscle graft chamber as depicted in FIG. 4. Referring to FIG. 4, nerve fibers (axons) from the amputated nerve 32 (encased in a soft nerve cuff 34) regenerate through very small tubular structures (microtubes) 36 that are open on both ends. In traversing the microtube array structure, the regenerating nerve fibers separate to a high degree so that one or only a few fibers grow into each of the individual microtubes within the array. Each microtube contains electrode contacts 40 to enable the fibers within the tube to be recorded from and/or electrically activated. For example, two contacts on each end of a microtube may serve as either recording reference electrodes or as stimulation current return electrodes. Other electrode embodiments include biochemically functionalized electrodes, optical sensors of action potentials (sometimes called “optodes”), or biochemical sensors that have optical readout instead of electrical readout, capacitive sensors for action potentials, or biochemical sensor readout using capacitive measures.

The cross-sectional circumference of the microtubes can be circular, hexagonal, triangular, trapezoidal or of any other plane closed geometry. While circular is the obvious choice for an appropriate shape because nerves are in general circular as well, it may be that another shape will provide a better environment, for example, because diffusion of nutrients and wastes could be enhanced in some way.

Referring still to FIG. 4, in a similar manner to the implementation using target tissues with the embodiment of FIG. 2, groups of microtubes 36 can also be connected to various target tissues such as nerve, muscle 38a, tendon, and skin 38b, separated out into individual larger tubes connected to and continuous with the microtube array. The target tissues can both enhance the health and function of the axons and segregate axons by function. Alternatively, or in addition, selective growth factors or other bioactive molecules (such as laminin, blood plasma or other neurotrophic chemicals) for segregating the axons can be used to coat the inside walls of selected microtubes or they can reside in a slow release polymer or gel, for example, in place of the target tissue. Target tissues can exist, e.g., as thin slices of the appropriate tissues, minced tissues, minced tissues in a nutrient gel, cell extracts from tissues or cultured tissues.

If the microtubes that the axons grow into are small, there will necessarily be a small number of axons within the microtube (e.g. 1-10), and thus will statistically more likely be from similar functional populations than larger groups of axons. By providing many such chambers, an entire peripheral nerve can thus be divided into small segments. Each microtube can independently be sensed for efferent information that would be useful for controlling a prosthesis for example, or stimulated to provide sensation through afferent axons.

Because peripheral nerve axons tend to be grouped together anatomically by function and target organ or tissue, it is probable that most axons within a particular microtube will be targeting the same anatomical muscle or the same type of sensors. When this occurs, the ideal situation is achieved. If these are efferent axons, the information can be used to control a particular muscle function in a prosthesis for example with no crosstalk with other sources. If afferent axons are present within the microtube, these axons can be activated by electrical stimulation to provide sensation. If a mixture occurs, then either function is possible, and may be chosen for particular needs of the prosthesis at any time, or simply assigned to a particular function.

The following is a more detailed description of one possible design. For most applications, many microtubes in an array within one structure would be advantageous. This would create as many information conduits as possible between an electrical system and the nervous system. This structure would have a vestibule at one end to hold the peripheral nerve in approximation to the entrance to the microtube array. The mechanical properties of this vestibule should match the peripheral nerve mechanical properties to reduce the possibility of neural damage from relative motions. In the middle, the structure of the microtube array would be placed to allow regenerating axons from the peripheral nerve stump to grow through. If target tissues are provided, they would be coupled to the end of the microtube array in several possible ways. The simplest would be to suture the targets to the end of the microtube arrays and allow the natural healing process time to stabilize and compartmentalize the tissues. Another technique would be to put relatively large tubes or somewhat flattened tubes, perhaps 3, each ⅓ of the area of the microtube assembly, and suture targets into each one. It may be important to produce targets that are in small, thin slabs to facilitate exchange of nutrients before the blood supply is re-established, and to provide a large surface area for ready access to the regenerating axons.

Microtube diameters may be from 10 um to 1 cm and range in length from 10 um to 2 cm, but preferably a few internodal distances to ensure that at least 1 Node of Ranvier (small active segment of a myelinated fiber) is contained within the microtube. Since regenerated mammalian nerves have internodal distances of about 200 um, tube lengths should be approximately 0.4-1 mm. Preferably, the microtubes may be 30-200 um in diameter (sufficiently large to perhaps allow small blood vessels to grow through, but sufficiently small to allow only a modest number of axons to grow through and thereby restrict interaction with a particular electrode). Non-myelinated fibers (pain and sympathetic) will also regenerate through the tubes but can be differentially stimulated by proper selection of stimulus parameters (amplitude, pulse duration and waveshape). Selective recording from myelinated vs non-myelinated fibers can also be accomplished, but by sorting the neural activity by waveshape and amplitude.

Electrodes can be fabricated on the bottom of the microtube. The electrical interconnects to the electrodes would be insulated, e.g., with LCP, thin silicone, Parylene, or other thin insulating, bioresistant, biocompatible material. LCP is a dimensionally stable, micro-machinable, biocompatible, chemical resistant, bioresistant material that has great promise as a long term implantable material for neuroprostheses.

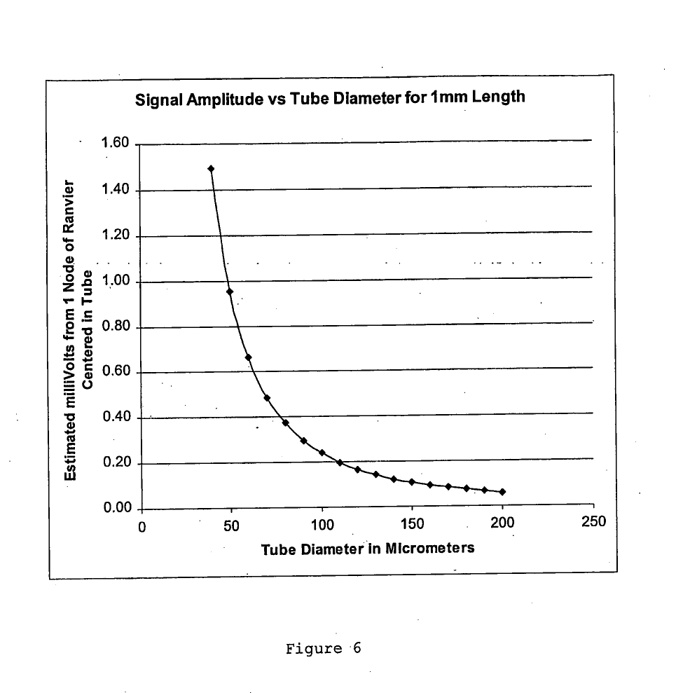

The 3 nanoampere signals or so that come out of myelinated axons then must flow through relatively high impedances created by the constricted space of the small tubes. This creates relatively large voltages that can be easily sensed for use in motor control of a prosthetic limb, for example. Referring to FIG. 6, a simple model was used to generate a graph of anticipated electrical potential maximum (signal amplitude) due to an action potential from a single axon versus tube diameter in a 1 mm long microtube.

To minimize noise, electrode contacts ideally would circumferentially cover the inner wall of a microtube over roughly the middle eighth or quarter of the microtube. This will maximize electrode surface area without causing a significant reduction in signal amplitude. The precise geometry can be optimized by straightforward modeling, trading off signal amplitude with electrode noise. However, a simple planar electrode on the surface of or embedded within the sidewall of a microtube would likely be sufficient since the signal levels will be high in any case. Electrode contact material ideally would be iridium oxide, but could almost as ideally be stainless steel, tantalum oxide, titanium oxide, titanium nitride, platinum, gold, or any other biocompatible, bioresistant, electrochemically friendly electrode material with even modest charge transfer capability (capacity to transfer charge without causing electrochemical degradation of the tissue or electrode).

Also because of the constricted space, application of a very small voltage for 10-100 uS can readily depolarize the axon membranes causing them to fire an action potential thereby enabling injection of prosthetic sensation to the amputee from sensors on a prosthesis. The low stimulation thresholds will be due to directly setting the extra-cellular potential by changing the potential on the electrode (approximately −20 mV to −1001V) with very little current flow because of the high impedance environment created by the small diameter insulated microtubes. It is advisable to use inverse parallel diodes on all electrodes to minimize the possibility of electrically damaging the axons within these microtubes. This change in external membrane potential will rapidly cause depolarization of the axon membrane at the Node of Ranvier thereby causing an action potential to propagate. Low stimulation thresholds have many significant advantages including relaxed design parameters for electrode geometry and materials, greatly reduced chance of neural damage from electrochemical generation of toxins, essentially infinite electrode lifetime, the possibility of using capacitive electrodes (even safer than noble metals or noble metal oxides), and the possibility of stimulating and recording on the same electrodes with minimal artifact.

A key element central to this invention is a means of fabricating the three-dimensional structures with integrated electronics, interconnects and electrodes. By using a dimensionally stable, biocompatible, bioresistant, chemically resistant, flexible polymer substrate, it is foreseeable that a device with several hundred or several thousand channels could be created and interfaced to a peripheral nerve.

It will be possible to fabricate the proposed microarray in several ways. Ideally, the method would take advantage of planar microtechnology to allow fabrication of such small, and so many structures on one substrate. One relatively simple approach would be to fabricate the structure in layers. For example, a planar substrate could be patterned with electrodes, interconnects, and a circuit layout. In the electrode area, sidewalls of tubes could be screen printed using silicone for example, or photolithographically defined by using a photo-polymer, or by casting on a material that is then later etched through a patterned top layer of photoresist. By stacking many of these, the backside of one layer would form the top of the microtubes of another, thereby creating the hollow tubular structures, with embedded electrodes. The drawback to this approach is that the many electrodes from one level would have to be interconnected in some way with the circuitry. While it can be imagined that this could be accomplished by having the circuit interconnected with a micro-ribbon cable to each set of microtubes, cutting them from a monolithic substrate (to avoid the issue of interconnecting and insulating so many micro-interconnects) there would be a jumble of micro-ribbon cables if more than a few layers were stacked. Then, if several of the microtube assemblies were to be interconnected to one circuit, the jumble of ribbon cables may be difficult to handle though certainly not impossible.

An alternative might be to design circuits that interact with each layer individually, and then communicate with a master telemeter using, e.g., optical or electromagnetic coupling. The master telemeter would then transmit and receive the information through the skin.

Another approach would be to fabricate the three-dimensional structure as above, but rather than stacking the devices, roll them up so the backside of the substrate becomes the top of the microtubes, so looking at it from nerve entry end it would appear as a micro-chambered nautilus. The interconnects could become an issue in that the length of the microtubes will probably be small (˜1 mm or less), so for a one-level metal system, with hundreds of connections, the width of the substrate being rolled up might exceed the desired microtube length. Use of multilevel metals is an obvious solution, but an expensive choice that may incur significant reliability issues. The preferred approach would be to extend the substrate beyond the defined micro-tubes, to provide room for the interconnects. When the unit is rolled up, there would be a non-interrupted spiral channel interconnecting but orthogonal to the long axis of the microtubes. This spiral channel could be used for fixation of relatively thin sheets of target organs (perhaps as thin as 100 μm or less), which would provide thousands of target cells for neurons to grow into. If helpful, this spiral channel can be partitioned. An advantage of using very thin target tissues is that they will not degenerate as nutrients and oxygen can easily diffuse in until the blood supply is re-established, and wastes can diffuse out.

It may be advantageous to position electrodes on one side of the substrate and the relief pattern that can form the microtubes on the other side. By placing one layer on top of another, the electrode contacts on the backside of one layer become the electrode contacts for the microtubes underneath. Likewise, if the substrate is rolled into the chambered nautilus shape, the appropriate electrode/microtube relationship would be formed.

The microtubules could be coated with laminin or other bioactive molecules known to promote or maintain axon health and regeneration. Also, other bioactive molecules such as nerve growth factors, possibly in a time release polymer, could be used in the microtubes, the spiral channel, or the holders for the tissue targets.

It is preferable to use a mechanical strain relieving nerve cuff fabricated usually from soft silicone elastomers. This strain relieving nerve cuff can also have electrodes located within it in order to activate the enclosed nerve axons by normal electrical stimulation methods or to record from the axons when they are active. A peripheral nerve fascicle or fascicles can be inserted or suctioned into this silicone vestibule and sutured or glued in place with standard microsurgical techniques. For large, multi-fasciculated nerves, it may be advantageous to use several microtube arrays, one on each fascicle. Typically, this might be done using standard fascicular dissection followed by suction or insertion of the fascicle or fascicles into the vestibules of the microtube array assembly.

Virtually any biocompatible, bioresistant, flexible, dimensionally stable, chemically resistant, tough, substrate could be used for the described processes above. Obvious ones would be another polymer, or metals such as ultra thin titanium or tantalum both of which can be oxidized to produce a reliable insulating layer that metal patterns can be deposited onto and which could be rolled up or stacked as previously described. Non-oxidizable metals could also be used but an insulating layer would first have to be coated onto them.

Another alternative to LCP, which may someday allow full integration of all electrical systems, would be to use micromachined silicon integrated circuit technology similar to that being developed at the University of Michigan. This technology allows fabrication of structures similar to those described above that have advanced CMOS integrated circuits as well as micromachined structures and electrode arrays on substrates that are only a few microns thick. When silicon is thinned to this level, it becomes very flexible in the thin dimension. Thus, walls could be patterned onto the silicon substrate using screen printed silicone for example (which bonds well to silicon and also protects it from the body environment), or by direct micromachining. Then the device could be rolled up to form the chambered nautilus structure previously described. However, silicon this thin is very fragile, and it is not known if this single crystal material could withstand the permanent bend without eventually failing. It could be reinforced by laminating it to the LCP substrate material (likely with a silicone bonding layer) which could have significant advantages. This final structure then would have very few issues with dimensions since sub-micron linewidths are now possible, so a very high channel count assembly could be fabricated with integrated electronics for the telemetry.

In particularly preferred embodiments, the long term bi-directional axon-electronic communication system according to the invention is equipped for enhanced fascicular specificity and the possibility of providing sensory feedback. As applied to, e.g., the nerve-muscle graft chamber embodiment, the individual fascicles of the transected nerve are mechanically divided as much as possible so that the individual fascicles can be introduced into separate vestibules on the device (refer again to FIG. 2), which serves to preserve their physical separation. Fascicles can also be divided manually to provide further subdivisions. The nerve fibers (axons) within each vestibule are then expected to grow into the respective compartments which are seeded with appropriate target tissues that act to both enhance the robustness (magnitude) of the neural growth, and to provide a biologically stable environment so that the regenerated nerve fibers remain healthy indefinitely. For example, muscle tissue is the preferred target tissue for regenerating motor (efferent) nerve fibers, while cutaneous tissue is a suitable target for tactile sensory (afferent) nerve fibers.

With a more advanced embodiment, the target tissues used in each chamber could be very specific such as a single class of sensory ending (i.e., joint receptor, muscle spindle receptor, tendon organ, Paccinian corpuscle etc.).

Referring now to FIG. 5, once the sensory afferents have re-established connections with the appropriate target tissues 58, it would be possible to electrically activate those sensory afferent nerves via the electrodes 60 in each tissue compartment 56 to induce sensations for sensory feedback. In addition, it might also prove to be advantageous to provide a mechanical stimulus to the specific tissue that is within the chambers. Thus, a micro-actuator 62 could be used to apply a stretch to the muscle tissue 58 and activate the sensory innervated muscle spindle organs to induce the perception of joint motion.

The rationale for applying mechanical stimulation to the muscle slices within the chamber channels derives from the fact that it is generally accepted that proprioceptive information about limb position is partially derived from the activity of muscle spindle receptor afferents. Basically, the natural proprioceptive system works by computing the vectoral sum of the discharges from all of the muscle spindle afferents that attach to a particular joint. For example, if one considers the flexion-extension axis of the human wrist joint, a flexion movement occurs due to contraction of the flexor muscle and relaxation of the extensor muscle. This contraction of the flexor muscle acts to silence or reduce the activity from its intrinsic stretch receptors and at the same time, the flexion movement of the joint serves to stretch the extensor muscle which is located at the opposite side of the joint. This stretching of the extensor muscle causes its stretch (spindle) receptors to increase their discharge (because the preferred stimulus for spindle receptors is stretch). The human brain then compares the ongoing neural activity arriving from the flexor and extensor muscle spindle afferents and because the extensor activity has increased and the flexor activity has decreased, the sensory experience that results is one of wrist movement in the flexion direction.

Many below elbow amputees could use contractions of paired muscles which formerly (before the amputation) acted as antagonists across the wrist joint for example to control flexion and extension of a prosthesis wrist joint. Because the muscles are no longer tied to the joint, a contraction (and shortening) of one of the pair of muscles has no means of stretching the antagonist muscle, and thus no meaningful changes in the relative activities of the muscles stretch receptors occurs and the amputee doesn't perceive a meaningful and accurate sensation regarding the prosthesis wrist joint.

To implement the provision of proprioceptive information, micro-servo actuators are attached to the pieces of muscle tissue that are present within each compartment of the nerve-muscle graft chamber. The servo actuators would serve to regulate the amount of tension present within the muscle tissue (and so cause it to be stretched or to relax) so that its spindle receptors would increase or decrease their discharge activity, respectively, as desired. The amount of the increase or decrease would be adjusted as needed in order to produce sensory experiences that tracked the changes in position of the relevant prosthesis joint. It may also be possible to vibrate the target muscles or to electrically stimulate them to activate the stretch sensors, thereby causing proprioception.

This aspect of the invention can also be applied to such systems as described above which involve any combinations of proprioceptive or tactile afferent nerve fibers which naturally innervate or innervate by nerve grafting techniques, any target tissues derived through other means either naturally derived, synthesized or grown from precursor or other cells.

In the case of cutaneous tissue, the micro-actuators would be fastened to the target tissue or placed in apposition to it to be able to mechanically deform it so as to influence the discharge activity of tactile sensory end organs that are contained in that target tissue. Thus, stretch, vibration or pressure could be applied to target tissue that contained cutaneous mechanoreceptor afferents. For example, vibration applied to Paccinian receptors in a cutaneous innervated chamber slot would evoke a vibratory sensation, and compression applied to Merkle receptors would evoke a sensation of cutaneous pressure.

The following describes one envisioned use of the system of the invention, and, in particular, the embodiment according to FIG. 4, for an above elbow amputee. This description assumes that one axon-electronic communication system will be used for each fascicle of the median nerve. During surgical modeling of the residual stump of the amputated arm, the peripheral nerves are mobilized from the connective tissue and moved to deeper, protective locations away from the end of the stump but near the humerus. The electronics module with attached communication systems is mounted to the bone to provide a mechanically stable location for the interfaces. Short micro-ribbon cables that connect the communication systems to the electronics module are arranged in a convenient order to facilitate insertion of the median nerve fascicles.

Thin slices of target tissues are cut from suitable muscle and skin of the patient, shaped to fit the target tissue receptacles, and inserted into the receptacles. To maintain the tissues in the receptacles, a small drop of plasma, a biocompatible gel (e.g., polyethylene glycol), fibrin glue, sutures, or a perforated cap can be applied. It is also likely that during the molding process for the tissue target chambers, small protrusions of silicone can be included to provide sufficient friction to keep the tissue targets in place once inserted without impeding nutrient, oxygen, waste exchange, re-innervation and re-vascularization.

The fascicles of the median nerve stump are then dissected free of the epineurium that holds them together. Each fascicle is sutured into a soft nerve cuff by passing a small (8-0 to 10-0) nylon suture through the cuff and through the perineurium. The position of the end of the fascicle is a few millimeters from the microtube array within the nerve cuff to allow regenerating axons to migrate along trophic factor gradients toward the specific microtubes that are associated with their corresponding target tissues. The remaining damaged tissues are surgically repaired or modified as needed to form a healthy stump, to complete the surgical procedure.

Once the regenerating axons reach the microtubes, they will grow through, and reinnervate the appropriate cells within the target tissues. Revascularization of the target tissues, essential for long term survival, function and maturation of the target cell-axon pairs, will occur by regeneration and sprouting of small vessels and capillaries (within the nerve stump) through the microtube array or through somewhat larger “vessel” tubes included in the micro-tube array, or by sprouting of external nearby vessels that grow directly into the target tissue chambers, or all of the above.

Once axons have regenerated through the interface and have recovered function, it will be possible to record relatively large signals from the efferent axons via the electrodes located within the tube. Also, it will be possible to trigger action potentials in the axons by passing small currents through the electrodes. Recorded signals from the efferent axons will be telemetered optically across the skin and then processed by a microcontroller for use, e.g., in controlling one or more motor drives within the mechanical prosthetic arm. These signals can also be processed and directly used to control a computer through a serial port or the keyboard port, thereby obviating the need for mechanical typing to control the computer. The computer, of course, can then be used to operate anything that can be electrically controlled.

By telemetering data into the implanted receiver of the communication system, one or more electrodes can be selected to stimulate axons within particular micro-tubes to generate perception of sensation. The information used to generate the perceptions would come from sensors embedded in the prosthesis. This information would be used to encode the sensations in physiologically relevant pulse sequences that would be used to properly activate the axons for “natural” sensation. By providing stimulation relevant to the prosthetic limb joint positions to afferent fibers associated with proprioception, awareness of the position of the limb in space can be provided to the amputee.

Once the stump has healed sufficiently, the prosthetic limb can be fitted to the stump. Placed within the prosthetic limb would be receivers for receiving information from the communication system and transmitters for sending power and data to the communication system. While this could also be accomplished through a percutaneous connection with wires, use of telemetry would be more acceptable to the patient as well as safer. An option would be to fit an electrical interface that could exchange information between the residual nerve and virtually any electrically interfaced machine. Of course, both could be combined—a mechanical prosthetic limb with a small plug port or secondary telemeter to also allow direct exchange of information between the communication system of the invention and electrically interfaced machines.

Use of the communication system of the invention in amputees of any type is a straightforward modification of the above description, with only the location of the implant being modified. The system of the invention may also find application in spinal cord injury. By transecting a paretic peripheral nerve whose motor neurons are located below the level of spinal cord injury, a communication system according to the invention can be installed as for an amputee with the following modifications. Instead of using target tissues and target chambers, the system described above would be modified by replacing the target chambers with a simple distal nerve cuff. The implant procedure would then involve transection of the peripheral nerve, suturing the proximal fascicles into the proximal end nerve cuff of the communication system, and suturing the corresponding distal fascicle into the distal end nerve cuff. The distal fascicle would then serve as an ideal trophic influence on the regenerating nerve which will then grow through the microtubes and reinnervate the original tissues.

A similar procedure could be accomplished in the spinal cord descending tracts at the level of spinal cord injury by insertion of a microtube array at the proximal end of the damaged motor tracts. By treatment of the area and implant with biochemicals designed to retard formation of a glial scar for sufficient time to allow regeneration of the descending motor axons through the microtube array, an interface with motor efferents of that were interrupted by the injury could be accomplished. Information from these efferents could then be used to directly communicate with a computer and computer interfaced machines and assistive devices, or to re-animate paretic muscles for movement and control of defecation, urination, breathing, posture, etc as described below.

Reanimation of paretic muscles can be accomplished by first receiving motor control information from the descending tracts of the spinal cord through a microtube array that has been interfaced with the regenerating axons before gliosis sets in. This information would then be translated into a series of electrical pulses used to generate action potentials in the axons going to the appropriate (intended) muscles that have regenerated through a system according to the invention. These action potentials will cause muscle contraction similar the intended contraction. Proprioception and cutaneous sensations can be returned to the central nervous system using electrode arrays implanted in the somatosensory cortex to complete the system.

In order to facilitate installation of a system according to the invention in amputees, a kit can be prepared that contains a communication system suitable for interfacing with a specified number of nerve fascicles (one or more, perhaps up to 20). A standard orthopedic hand surgeon's tool set containing all the generally used micro-tools for nerve repair and tissue reconstruction and an amputation pack can also be included.

A suction fitting that mates with the distal end of the communication system can also be included in the kit. This fitting is used advantageously to draw the peripheral nerve into the proximal cuff as sutures are tightened. Small cross-bars, cast within the proximal cuff can be used to set the distance from the end of the nerve to the microtube array without significantly impeding the progression of regenerating axons.

Microbiopsy punches or hollow drills are included to take “core” samples of muscle and skin (preferably from areas rich in sensors such as fingertips). These are brought to an opening in the distal end of the microtube array and inserted by pushing a plunger into the hollow punch or drill and ejecting the tissue into the chamber of a microtube. If tissue homogenate is used, the core samples would first be finely chopped or disrupted biochemically, and the resulting slurry could be simply injected into the tissue chamber. The slurry may become a gel at normal body temperature or in response to treatment with a salt such as calcium chloride or other, depending on the composition. A chamber can have small fibrous protrusions of silicone or other soft polymer or material pointed “into” the chamber to prevent the injected or ejected tissue samples from easily escaping, while still allowing free communication with the surrounding fluids for nutrients and waste removal.

In order to insert a large number of sensory and motor targets, a multi-site injector/ejector can be constructed that mates precisely with the physical layout of the tissue target chambers of the system. By keying the device to the layout of the chambers, muscle targets can be injected into specific chambers while sensory targets can be injected into other chambers.

The system implant itself consists of a plurality of microtube regeneration arrays with microcable connections to the central controller/transceiver. The unit has a power source such as light through photocells, RF, ultrasound, temperature, or battery (preferably rechargeable), fuel cell, etc. This power can be used to operate the implant amplifiers, multiplexers, encoders, decoders stimulators and transmitter.

Preferably, the entire kit is sterile packaged, with the implant being submerged in, e.g., a sterile physiological saline solution to protect the fragile biochemicals, such as laminin, that may be attached to critical implant surfaces, e.g., to promote biocompatibility, axon regeneration, and to retard fibroblast and glial scar formation until the axons have regenerated.

In summary, the long term bi-directional axon-electronic communication system according to the invention also has application, e.g., to lower extremity prostheses where the EMG signals can be used to control the action of a powered ankle or powered knee joint or to control a locking mechanism for prosthesis knee or ankle joint, for example. Such devices would be particularly useful for amputated nerve interface work, but could also find application as an interface for nerves of paralyzed individuals, e.g., as a spinal cord interface, or perhaps even a cortical interface. Other applications can include anything that can be controlled by a computer including wheel chair control or environmental control such as light switches, appliances or powered door openers, for example. This general utility can be understood by knowing that computers can control any electrically controllable machine, and that the bi-directional nerve-tissue interface can, with proper encoding, communicate with any computer,

### REFERENCES

Carlson B M., and Gutmann E. (1.972), Development of contractile properties of minced muscle regenerates in the rat. Exp. Neurology, 36:239-249.
Carlson B M, (1972), The regeneration of minced muscles. Monogr Dev Biol. 1972; 4:1-128. Review.
Charge Sophie B. P. and Rudnicki M. A. (2004), Cellular and molecular regulation of muscle regeneration, Physiol Rev. 84:209-238.
Edell D. J. (1980), Development of a Chronic Neuroelectric Interface, in Biomedical Engineering, University of California: Davis.
Edell D. J., Churchill J. N. and Gourley I. M. (1982), Biocompatibility of a silicon based peripheral nerve electrode. Biomat Med Dev Art Org, 10(2):103-122.
Edell D. J. (1986), A peripheral nerve transducer for amputees: long term multichannel recordings from rabbit peripheral nerves. IEEE Trans. BME, 33: 203-213.
Goodwin G. M., McCloskey D. I. and Matthews P. B. C. (1972), The contribution of muscle afferents to kinesthesia shown by vibration-induced illusions of movement and by effects of paralyzing joint afferents, Brain 95:326-329.
Hoffer J. A. and Kallesøe K. (2000), ‘How to use nerve cuffs to stimulate, record or modulate neural activity’. Chapter 5, In: Neural prostheses for restoration of sensory and motor function, K. A. Moxon and J. K. Chapin Eds., CRC Press, Boca Raton, pp 139-175.
Jabaley, M. E. (1991), Internal topography of peripheral nerves as related to repair, pp 231-240, In: ‘Operative Nerve Repair and Reconstruction, Gelberman, R. H., ed., Lippincott, Philadelphia.
Kovacs G. T., Storment C. W., Halks-Miller M., Belczynski C. R., Santina C. D., Lewis E. R. and Maluf N. I. (1994), Silicon substrate microelectrode arrays for parallel recording of neural activity in peripheral and cranial nerves, IEEE Trans. BME, 41:567-577.
Kuiken T. A., Lowery M. M. and Stoykov N. S. (2003), The effect of subcutaneous fat on myoelectric signal amplitude and cross-talk. Prosthet Orthot Int., 27(1):48-54.
Kuiken, T. (2004), Consideration of nerve-muscle grafts to improve the control of artificial arms. J. Technology and Disability. (Submitted)
Kuiken T., Stoykov N., Lowery M. and Taflove A. (2004a), Finite element analysis of EMG signals to improve the control of myoelectric prostheses. 10^{th} World Congress of ISPO, Glasgou, UK.
Lowery M. M., Stoykov N. S., and Kuiken T. A. (2003), Independence of myoelectric control signals examined using a surface EMG model. IEEE Trans Biomed Eng. 50(6):789-93.
Lowery M. M., Stoykov N. S., Taflove A., and Kuiken T. A. (2002) A multiple-layer finite-element model of the surface EMG signal. IEEE Trans. BME., 49:446-54.
Malagodi, M. S., Horch, K. W. and Schoenberg A. A. (1989) An intrafascicular electrode for recording of action potentials in peripheral nerves. Annals of BME, 17:397-410.
McNaughton, T. G. and Horch, K. W. (1996) Metallized polymer fibers as leadwires and intrafascicular microelectrodes. Journal of Neuroscience Methods, 70:103-110.
Matjacic, Z., Jensen, P. L., Riso, R. R:, Voigt, M., Sinkjaer, T. and Bajd, T. (1999) “A Two-Dimensional Electrocutaneous Cognitive Feedback System for Use in Paraplegic Standing”. Journal of Biomechanics, June.
Mong F. F. (1977), Histological and histochemical studies on the nervous influence on minced muscle regeneration of triceps surae of the rat. J. Morphol. 151:451-62.
Navarro, X., et al., Stimulation and recording from regenerated peripheral nerves through polyimide sieve electrodes. J Peripher Nerv Syst, 1998. 3(2): p. 91-101.
Phillips G D, Lu D Y, Mitashov V I, Carlson B M. (1987), Survival of myogenic cells in freely grafted rat rectus femoris and extensor digitorum longus muscles, Am J. Anat. 1987 December; 180(4):365-72.
Riso, R. R. (1988), “Sensory Augmentation for Enhanced Control of FNS Systems, Chapter 17, pp 253-271, In Ergonomics in Rehabilitation, A. Mital, Ed., Taylor Francis, London.
Riso, R. R., Ignagni, A. R. and Keith, M. W.: (1999), Electrocutaneous Sensations Elicited Using Subdermally Located Electrodes, Automedica, 11:25-42.
Riso, R. R. (1999), Strategies for providing upper extremity amputees with tactile and hand position feedback—Moving closer to the bionic arm, Technology and Health Care, 16:401-409.
Riso, R., Dalmose, A., Schuttler, M., and Stieglitz, T. (2000), Activation of Muscles in the Pig Forelimb Using a Large Diameter Multipolar Nerve Cuff Installed on the Radial Nerve in the Axilla, Proc. of the 5^{th} Ann Conf. of the International Functional Electrical Stimulation Soc., p 272-275, Aalborg, DK, June.
Roldan, L., Riso, R. R., and Sepulvida, F. (2004) Neural Network Recognition of Natural Intramuscular EMG Pattern Recognition System for Multi Modal Grasp Control of a Below-elbow Prosthesis”, Submitted to IEEE Trans on Rehabilitation Engineering.
Roldan, L., and Riso, R. R. (1999) Natural Control of Key Grip and Precision Grip Movements for a Myoelectric Prostheses, Proceedings of the Myoelectric Control Conference, University of New Brunswick, Fredrickton, Canada, July.
Roll J. P. and Gilhodes J. C. (1995), Proprioceptive sensory codes mediating movement trajectory perception: human hand vibration-induced drawing illusions, Can. J. Physiol. Pharm. 73:295-304.
Rutten, W. L., Smit J. P., Frieswijk, T. A., Bielen, J. A., Brouwer, A. L., Buitenweg, J. R., and Heida, C. (1999) Neuro-electronic interfacing with multielectrode arrays. IEEE Eng Med Biology Mag. 18:47-55.
Schuettler, M., Stieglitz, T., Keller, R., Meyer J.-U. and Riso, R. R. (1999), A hybrid cuff electrode for functional electrostimulation of large peripheral nerves”, Medical and Biological Engineering and Computing, 37(2):792-793.
Vedung S., Ulfendahl H. and Hakelius L. (1983), Freely transplanted peroneus longus muscle in the cat reinnervated by a single nerve: physiological properties. Scand J. Plast. Reconstr. Surg. 17:1-6
Wallman L., Levinsson A., Schouenborg J., Holmberg H., Montelius L., Danielsen N., Laurell T. [1999], Perforated silicon nerve chip with doped registration electrodes; in vitro performance and in vivo operation. IEEE Trans Biomed Eng. 46:1065-73.
Wallman, L., Yanzhen Z., Laurell T. and Danielsen N., (2001), The geometric design of micro-machined silicon sieve electrodes influences functional nerve regeneration, 22(10):1187-1193.
Wells, M. R., Vaidya, U., Ricci, J. L., Christie, C. (2001), A neuromuscular platform to extract electrophysiological signals from lesioned nerves: a technical note, J. Rehabilitation Res. and Dev. 38: (No. 4).
Yashida, K and Riso, R., (2003) “Peripheral nerve Recording Techniques and Methods”, Chapter 2.4, In Neuroprosthetics—Theory and Practice, K. W. Horch and G. Dhillon (Eds), World Scientific.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A long term bi-directional axon-electronic communication system comprising:
a nerve cuff for an input nerve;
a receptacle connected to said nerve cuff, said receptacle comprising one or more compartments for receiving individual axons, bundles of axons or fascicles of said input nerve;
a target tissue or plurality of target tissues for interfacing with said individual axons, bundles of axons or fascicles;
one or more sites in said receptacle for bi-directional communication to or from said individual axons, bundles of axons, fascicles, target tissue or plurality of target tissues; and
a bi-directional electrical, biochemical or physical communication pathway for stimulating, or receiving a signal from, said individual axons, bundles of axons, fascicles, target tissue or plurality of target tissues.

2. The communication system of claim 1, further comprising a telemetry system for transmitting a stimulating signal to said one or more sites or for transmitting a signal received from said one or more sites to a separate device.

3. The communication system of claim 2, wherein said telemetry system is configured for communicating with a separate device positioned externally to a patient using said communication system.

4. The communication system of claim 2, wherein said telemetry system is configured for communicating with a separate device positioned internally within a patient using said communication system.

5. The communication system of claim 1, wherein said input nerve is a peripheral nerve.

6. The communication system of claim 1, wherein said input nerve is a central nerve.

7. The communication system of claim 1, wherein said receptacle comprises channel compartments in a microdevice.

8. The communication system of claim 1, wherein said receptacle comprises individual microtubes.

9. The communication system of claim 8, wherein one or more of said microtubes has an interior coating.

10. The communication system of claim 9, wherein said interior coating comprises laminin or blood plasma.

11. The communication system of claim 1, wherein said target tissue is muscle or skin.

12. The communication system of claim 1, wherein said target tissue is an organ.

13. The communication system of claim 1, wherein said target tissue is selected from the group consisting of slivers of tissue, cells from disrupted tissue, minced tissue containing viable cells, and cells or minced tissue in a carrier material such as a gel.

14. The communication system of claim 1, wherein said one or more sites are electrodes.

15. The communication system of claim 1, wherein said one or more sites are optical sensors or biochemical sensors.

16. A kit for installing a long term bi-directional axon-electronic communication system, said kit comprising:
a nerve cuff for supporting an input nerve;
a tool for separating said input nerve into individual axons, bundles of axons or fascicles;
a receptacle to connect to said nerve cuff, said receptacle comprising one or more compartments for receiving individual axons, bundles of axons or fascicles of said input nerve; and
one or more sites in said receptacle for bi-directional communication to or from said individual axons, bundles of axons, fascicles, target tissue or plurality of target tissues.
